(19) United States
(12) Patent Application Publication
(10) Pub. No.: US 2004/0109825 A1
(43) Pub. Date: Jun. 10, 2004

(54) Topical patch preparation kit containing a delayed-type hypersensitivity inducer

(76) Inventors: Jutaro Shudo, San Jose, CA (US); Ichiro Mori, San Jose, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/675,789

(22) Filed: Sep. 29, 2003

Related U.S. Application Data
(60) Provisional application No. 60/420,225, filed on Oct. 21, 2002.

(51) Int. Cl. A61K 49/00

(52) U.S. Cl. 424/10.1

(57) ABSTRACT

Elevated temperature storage stable fluid compositions of a delayed-type hypersensitivity inducer, e.g., DNCB, and methods for using the same are provided. In many embodiments, the subject compositions are present in a sealed container and housed together with an unloaded topical patch preparation, e.g., in a kit format. In using the subject compositions, the fluid composition is applied to an unloaded topical patch preparation, which preparation is then topically applied. The present invention finds use in a variety of applications where the administration of a delayed-type hypersensitivity inducer is desired, and is particularly suited for use in the treatment of HIV associated disease conditions, e.g., AIDS.

### CROSS-REFERENCE TO RELATED APPLICATIONS

Pursuant to 35 U.S.C. § 119 (e), this application claims priority to the filing date of the U.S. Provisional Patent Application Serial No. 60/420,225 filed Oct. 21, 2002; the disclosure of which is herein incorporated by reference.

### INTRODUCTION

1. Field of the Invention

The present invention pertains delayed-type hypersensitivity-inducing agents.

2. Description of the Background Art

The number of Human Immunodeficiency Virus (HIV) patients worldwide has been increasing rapidly in recent years, and is said to be approximately 33(40) million (WHO(UNAIDS); end of 1998(2001). Against this backdrop, there is a rush to develop a vaccine for HIV. However, because of the mutation of the configuration of the virus following infection, a feature of HIV, an accurate vaccine has not yet been found. In addition, although many therapeutic medications for HIV have been developed, none completely cure HIV. Furthermore, current AIDS drugs (protease inhibitors, non-nucleoside reverse transcriptase inhibitors, nucleoside reverse transcriptase inhibitors, etc.) employ complex techniques. Long-term administration of these agents causes patients to suffer persistent adverse events, such as anemia, peripheral neuritis, pancreatitis, nausea, and headaches. Also, the possibility of long-term administration resulting in drug resistance cannot be ruled out. Yet another disadvantage of current treatment modalities is cost, in that current therapeutic medications for HIV are extremely expensive, often ranging between $15,000 to $20,000 per person per year, which necessarily limits patient access.

One type of agent that represents an effective alternative to current HIV treatment modalities is the delayed-type hypersensitivity (DTH) inducing agent, which type of agent has been researched as an immunomodulator that elicits immunological response in HIV patients by increasing the activity of the immune system cells in the body. Delayed-type hypersensitivity inducers are substances that induce Type 4 hypersensitivity when they come into contact with human skin, and they include trinitrobenzene sulfonic acid, picryl chloride(PC), 2,4-dinitrofluorobenzene(DNFB), and 1-chloro-2,4-dinitrobenzene (DNCB). Of these, DNCB has been widely used in the treatment of HIV and in immunological research.

DNCB was discovered in Germany before World War II. Research conducted in the 1950s in the United States demonstrated that DNCB is not carcinogenic. Later, in the 1970s, safety research was conducted in various types of animals. DNCB is generally known to be a powerful, delayed allergy-inducing skin irritant in humans, and is used in, among other things, immunological tests of skin diseases. Research on DNCB therapy in HIV patients began slowly from the middle of the 1980s, and research on DNCB therapy in HIV patients was conducted in the first half of the 1990s, from which DNCB was claimed to be effective for treating HIV. However, this claim was not proved. In the latter half of the 1990s, the development of PCR analysis technology began to confirm the efficacy of DNCB in HIV patients. In addition, DNCB was also previously investigated as a possible treatment for cancer: tests were conducted in which DNCB was applied locally to induce a delayed allergic reaction and thereby utilize its immunity inducing capabilities. However, these findings have not been put to practical use. Furthermore, DNCB has been used in, among other things, the treatment of warts.

A method for using DNCB in HIV patients that has been employed in recent years has been to dissolve the DNCB in an acetone solvent and impregnate a gauze-like cloth with the resulting product and apply this to the skin. This topical preparation is then dried, covered and left to stand for several hours (typically at least 8 hours). This long application time means that an HIV patient would be restricted for at least 8 hours, a fairly long time, which would prevent that person from leading the same lifestyle as a healthy person. Although this method provides for application of DNCB it is difficult to control the dosage and requires some time until the onset of efficacy.

Although DNCB may be utilized for the purposes described above, many times the environment in which the topical patches may be stored may lead to degradation of the effectiveness of the DNCB. It has been discovered that DNCB itself is unstable in a dissolved state, and even more unstable at high temperatures. In many countries where DNCB topical patches may be employed proper storage facilities are not available, and therefore the topical patch will no longer be effective due to degradation of the DNCB because of improper storage. There is considerable interest, therefore, in the development of a topical DTH inducing agent dosage form that is storage stable under a wide variety of conditions, including elevated temperature conditions.

Relevant Literature

References of interest include: Stricker et al. Dendritic cells and dinitrochlorobenzene (DNCB): A new treatment approach to AIDS. Immunol Letters 1991;29:191-196; Stricker et al. Pilot study of topical dinitrochlorobenzene (DNCB) in human immuno deficiency virus infection. Immunol Letters 1993;36: 1-6; Stricker et al. Topical dinitrochlorobenzene in HIV disease. J Am Acad Dermatol 1993;28:796-797; Stricker et al. Clinical and immunologic evaluation of HIV-infected patients treated with dinitrochlorobenzene (DNCB). J Am Acad Dermatol 1994;31:462-466; Stricker R B, Goldberg B, Mills L B, Epstein W L. Improved results of delayed-type hypersensitivity skin testing in HIV-infected patients treated with topical dinitrochlorobenzene(DNCB). J Am Acad Dermatol 1995;33:608-611; Stricker & Goldberg. Safety of topical dinitrochlorobenzene. Lancet 1995;346:1293; Stricker et al. Improved results of delayed-type hypersensitivity skin testing in HIV-infected patients treated with topical dinitrochlorobenzene. J Am Acad Dermatol 1996;35:491-493; Stricker et al. Decrease in viral load associated with topical dinitrochlorobenzene therapy in HIV disease. Res Virol 1997;148:343-348; Traub et al. Topical immune modulation with dinitrochlorobenzene (DNCB) in HIV disease: A controlled trial from Brazil. Dermatology 1997;195:369-373; Stricker et al. Topical immune modulation (TIM): A novel approach to the immunotherapy of systemic disease. Immunol Letters 1997;59:145-150; Oracion et al. DNCB treatment of HIV-infected patients leads to beneficial immunologic outcomes, reduced viral load, and improved measures of quality-of-life. J Invest Dermatol 1998; 110:476.

### SUMMARY OF THE INVENTION

Elevated temperature storage stable fluid compositions of a delayed-type hypersensitivity (DTH) inducer, e.g., DNCB, and methods for using the same are provided. In many embodiments, the subject compositions are present in a sealed container and housed together with an unloaded topical patch preparation, e.g., in a kit format. In using the subject compositions, the fluid composition is applied to an unloaded topical patch preparation, which preparation is then topically applied. The present invention finds use in a variety of applications where the administration of a delayed-type hypersensitivity inducer is desired, and is particularly suited for use in the treatment of HIV associated disease conditions, e.g., AIDS.

### BRIEF DESCRIPTION OF THE DRAWINGS

The invention will be more fully understood by reference to the following drawings, which are for illustrative purposes only.

FIGS. 1a and 1b provide a plan view of an exemplary container in accordance with the present invention; and

FIGS. 2a and 2b provide a cross-sectional side view of a topical patch in accordance with the present invention.

### DESCRIPTION OF THE SPECIFIC EMBODIMENTS

Elevated temperature storage stable fluid compositions of a delayed-type hypersensitivity inducer, e.g., DNCB, and methods for using the same are provided. In many embodiments, the subject compositions are present in a sealed container and housed together with an unloaded topical patch preparation, e.g., in a kit format. In using the subject compositions, the fluid composition is applied to an unloaded topical patch preparation, which preparation is then topically applied. The present invention finds use in a variety of applications where the administration of a delayed-type hypersensitivity inducer is desired, and is particularly suited for use in the treatment of HIV associated disease conditions, e.g., AIDS.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

It must be noted that, as used in this specification and the appended claims, the singular forms “a,” “an” and “the” include plural reference unless the context clearly dictates otherwise. Unless defined otherwise all technical and scientific terms used herein have the same meaning as commonly understood to one of ordinary skill in the art to which this invention belongs.

Where a range of values is provided, it is understood that each intervening value, to the tenth of the unit of the lower limit, unless the context clearly dictates otherwise, between the upper and lower limit of that range and any other stated or intervening value in that stated range, is encompassed within the invention. The upper and lower limits of these smaller ranges may independently be included in the smaller ranges, and such embodiments are also encompassed within the invention, subject to any specifically excluded limit in the stated range. Where the stated range includes one or both of the limits, ranges excluding either or both of those included limits are also included in the invention.

All publications mentioned herein are incorporated herein by reference for the purpose of describing and disclosing components that are described in the publications that might be used in connection with the presently described invention.

In further describing the subject invention, representative embodiments of the elevated temperature storage stable compositions are described first in greater detail, followed by a review of various container and/or kit configurations thereof, as well as a review of methods of using the subject compositions and representative applications in which the compositions find use.

Elevevated Temperature Storage Stable DTH Fluid Compositions

As summarized above, the subject invention provides elevated temperature storage stable fluid compositions of a delayed type hypersenstivity inducer agent (DTH). By “delayed-type hypersensitivity (DTH) inducers” is meant an immunomodulator that elicits immunological response in a subject, such as HIV patients, by increasing the activity of the immune system cells in the body. Delayed-type hypersensitivity inducers are substances that induce Type 4 hypersensitivity when they come into contact with human skin, and they include, but are not limited to: trinitrobenzene sulfonic acid, picryl chloride (PC), 2,4-dinitrofluorobenzene(DNFB), and 1-chloro-2,4-dinitrobenzene (DNCB). In many embodiments, the delayed-type hypersensitivity inducer is DNCB. By elevated temperature storage stable fluid composition is meant a fluid composition that is stable at temperatures in excess of at least about 35° C., typically at least about 38° C. and often at least about 40° C. for extended periods of time, e.g., at least about 30 days, usually at least about 90, at least about 180 days days, and sometimes at least about 1 year or more, e.g., 2.5 years, 5 years, etc. As the compositions are fluid compositions, they typically have a viscosity ranging from about 1 to about 1000 cps, usually from about 2to about 500 cps and more usually from about 2 to about 100 cps.

The subject fluid compositions include the DTH agent present in a suitable solvent that provides for the above-mentioned elevated temperature storage stable properties. In many embodiments, the solvent is a non-volatile organic solvent. Representative solvents that are suitable for use with the present invention include ester nonvolatile organic solvents and nonvolatile organic solvents that do not contain hydroxyl groups or metallic ions. Examples of representative ester nonvolatile solvents include, but are not limited to: diisopropl adipate, isopropyl myristate, and diethyl sebacate. Examples of representative nonvolatile organic solvents that do not contain hydroxyl groups or metallic ions include, but are not limited to: diethyltoluamide, crotamiton, and paraffin. It shall be understood that the above solvents should not be considered limiting in any manner in that many other solvents not referenced above may be utilized to practice the present invention, including solvents yet to be discovered.

The amount of DTH agent in the subject fluid compositions may necessarily vary depending on the nature of the particular solvent of a given composition and/or the particular type of DTH agent. However, in many embodiments, the amount of DTH present in the solvent ranges from about 0.01 to about 10.0% (w/w), usually from about 0.1 to about 5.0% (w/w) and more usually from about 0.2 to about 3.0% (w/w).

Sealed Containers

As indicated above, in many embodiments of the subject invention, the above described elevated temperature storage stable DTH fluid compositions are present in a sealed container, in which the fluid composition is present in a containment element that prevents the fluid composition from contacting the external environment of the container housing the composition.

The container may house or contain a single dosage or multiple dosages of a fluid composition, but generally contains a single dosage in many embodiments. Where the container is a single dosage container, the volume of fluid in the container typically ranges from about 0.2 ml to about 5 ml, usually from about 0.3 ml to about 3.5 ml and more usually from about 0.5 ml to about 2 ml.

The container may be constructed of a variety of different materials, so long as the material is inert to the fluid composition housed therein under conditions in which the container is storage, e.g., at elevated temperatures (as described above) and under conditions of varying humidity, e.g., from about 20% to about 95%, usually from about 30% to 80% (humidity). Examples of suitable materials include, but are not limited to: metallic materials, e.g., aluminum, and the like; polymeric or plastic materials, e.g., polyesters (such as polyethylene (PET) and polypropylene (PP)), and the like. It shall be understood the material examples above are to be considered exemplary and are not intended to be limiting in any manner.

As described above, the container may be constructed to have any shape, where the container in accordance with the present invention is typically designed to provide a container that prevents contact of the contents of the container with outside air, and can maintain a sealed state for a long period of time.

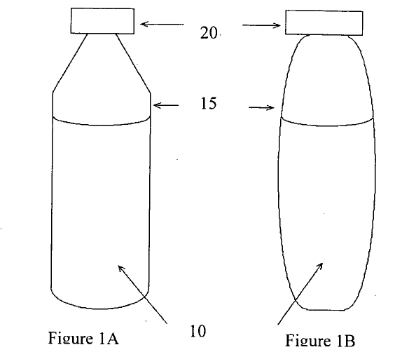

Referring now to FIGS. 1a and 1b (front and side views, respectively), there is shown an exemplary embodiment of a container in accordance with the present invention. As show in FIGS. 1a and 1b, the container 10 includes a generally tubular portion 15 and a removable sealing device (such as a cap) 20. The removable sealing device maybe integrally formed with the tubular portion 15, wherein the sealing device 20 may be removed through an action such as twisting or cutting. Alternatively, the removable sealing device 20 may a separate but engaged element, e.g., threadably engaged, to a portion of the tubular body to provide a fluid tight and an airtight seal.

In addition to the removable sealing device 20, it is contemplated that a secondary sealing device may be fixedly attached to the container, wherein the secondary sealing device is configured to be removable or penetrated to release the contents of the container. Wherein the secondary sealing device provides a supplemental or primary airtight seal between the contents of the container and the surrounding environment.

As indicated above, the representative container shown in FIGS. 1a and 1b is merely representative, and not provided to in any way be limiting with respect to the subject invention.

Kits

Also provided are kits that at least include an elevated temperature sensitive DTH fluid composition, as described above, where in many embodiments the fluid composition is present in a sealed container, as described above. In addition to the fluid composition, many representative embodiments of the subject kits also include an unloaded topical patch preparation.

The unloaded topical patch preparation may take a variety of different configurations, but typically includes at least a fluid retaining layer for receiving the DTH fluid composition and an adhesive layer for maintaining the patch in position once the patch is topically applied.

The fluid retaining layer may be made from any convenient material that is capable of retaining and then slowly releasing the fluid DTH composition in an acceptable manner. The fluid retaining layer is generally capable of holding a volume of the DTH composition that is at least about 0.1 ml, usually at least about 1 ml and sometimes at least about 5 ml. The fluid retaining layer may be made from any convenient material, where representative materials of interest include, but are not limited to: gauze or a nonwoven cloth, e.g., fabricated from PET(Polyethylene), PP(Polypropylene), and the like.

As indicated above, the unloaded topical patch preparation further includes an adhesive layer, where the adhesive layer serves to secure the topical patch to the topical location of a subject to which the patch has been applied during use. The adhesive layer may be made up of a number of different materials, so long as it provides for the adhesive function described above, where representative materials of interest include, but are not limited to: polymeric adhesive composition that are commonly employed in external preparations, e.g., styrene-isoprene-styrene (SIS) copolymers, styrene-butadiene-styrene (SBS) copolymers, etc.

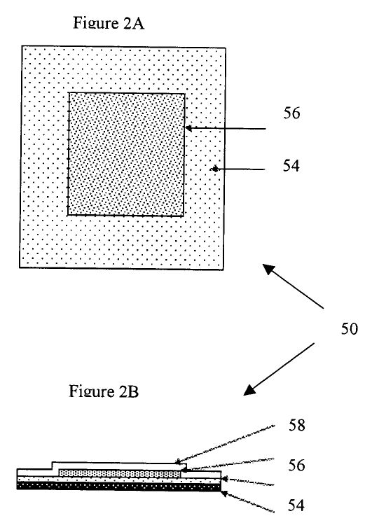

Referring now to FIGS. 2a and 2b (top and side views respectively) there is shown an exemplary embodiment of an unloaded topical patch preparation that may be present in kits according to the present invention. As shown in FIGS. 2a and 2b, the topical patch 50 includes a backing 52, an adhesive layer 54, a fluid composition retaining layer 56, and a release liner layer 58.

The representative topical patch depicted in FIGS. 2a and 2b has a rectangular or square configuration. The dimensions of the various layers may vary, and representative dimensions for each layer are provided below. With respect to the fluid retaining layer 56, in certain embodiments this layer has a length ranging from about 1 cm to about 7 cm, often from about 3 cm to about 6 cm; a width ranging from about 1 cm to about 7 cm, often from about 3 cm to about 6 cm; and a height ranging from about 0.2 mm to about 1.2 mm, often from about 0.5 mm to about 1 mm. With respect to the adhesive layer 54, in certain embodiments this layer has a length ranging from about 3 cm to about 9 cm, often from about 5 cm to about 8 cm; a width ranging from about 3 cm to about 9 cm, often from about 5 cm to about 8 cm; and a height ranging from about 0.01 mm to about 0.2 mm, often from about 0.03 mm to about 0.1 mm, and in many embodiments surrounds the periphery of the fluid retaining layer, as depicted in FIGS. 2a and 2b.

The backing layer 52 may be fabricated from a variety of different types of materials. When selecting a material to be utilized as a backing layer durability should be considered. The topical patch in accordance with the present invention is intended for use in inhospitable climates as well as generally mild climates. After application to a subject's skin, the topical patch may be subjected to a harsh environment. As such, a material that is durable under such conditions is also desirable. Furthermore, the backing 52 should be constructed of a material that is water resistant or waterproof, thereby enabling a user to bath or wash while wearing the topical patch in accordance with the present invention. The backing layer 52 is generally made of a flexible material which is capable of fitting in the movement of human body and includes, for example, various non-woven fabrics, woven fabrics, spandex, flannel, or a laminate of these materials with polyethylene film, polyethylene terephthalate film, polyvinyl chloride film, ethylene-vinyl acetate copolymer film, polyurethane film, and the like. With respect to the backing layer 52, in certain embodiments this layer has a length ranging from about 3 cm to about 9 cm, often from about 5 cm to about 8 cm; a width ranging from about 3 cm to about 9 cm, often from about 5 cm to about 8 cm; and a height ranging from about 0.02 mm to about 1 mm, often from about 0.03 mm to about 0.3 mm.

Also shown in FIG. 2b, also present is release liner or protective layer 58 that may cover the fluid retaining layer prior to loading with the fluid DTH composition. With respect to the protective layer 58, in certain embodiments this layer has a length ranging from about 3 cm to about 9 cm, often from about 5 cm to about 8 cm; a width ranging from about 3 cm to about 9 cm, often from about 5 cm to about 8 cm; and a height ranging from about 0.01 mm to about 0.5 mm, often from about 0.02 mm to about 0.2 mm. This layer may be fabricated from any convenient material, including the backing materials described above.

A protective layer (not shown) may be disposed over the release layer, wherein the protective layer is removed prior to use of the topical patch 50 in accordance with the methods of the present invention. As such, the topical patch component of the kit may be present in an individual pouch or analogous container.

The topical patch may be constructed using any known methods of construction. After fabrication the topical patch may be sealed within packaging. Examples of suitable packaging materials are packing materials including an aluminum layer or any other type of material that will protect the topical patch from contamination. A large number of topical patches may be sealed within a single packaging, though in a preferred embodiment, the topical patches are individually packaged.

In many embodiments of the subject kits, the fluid composition/container elements and the unloaded topical patch elements are further packaged in a kit containment element to make a single, easily handled unit, where the kit containment element, e.g., box or analogous structure, may or may not be an airtight container, e.g., to further preserve the composition of the patches and fluid composition until use.

The subject kits also generally include instructions for how to prepare or load the topical patches with the fluid DTH composition for use and/or how to use the patches for delivery of a delayed-type inducing agent to a host. In many embodiments, the instructions typically include information about how to prepare a topical patch for use, where to apply the patch, dosing schedules etc. In certain embodiments, the subject kits include instructions on how to use to the patched to treat a particular disease condition with a DTH inducing agent.

The instructions are generally recorded on a suitable recording medium or substrate. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Methods of Using the Subject Fluid Compositions

Also provided are methods of using the subject storage stable DTH fluid compositions. In general, the subject methods include the steps of providing a fluid composition of the present invention and then topically applying a sufficient or effective amount of the fluid composition to a subject or patient in need thereof.

In many embodiments, e.g., where the fluid composition is provided in a kit format as described above, the subject methods include the steps of preparing a topical patch preparation and then applying the topical patch preparation to a subject or patient in need thereof. In these embodiments, the topical patch is typically prepared by first opening a container that holds the fluid DTH composition and then depositing a sufficient volume of the DTH fluid composition on the fluid retaining domain or region of an unloaded topical patch preparation.

With respect to the representative configuration shown in FIGS. 1a,b and 2a,b, to prepare a topical patch from the components shown in these figures the first step is typically to remove the topical patch from any packaging that may be present, and then to remove the protective layer 58 from the topical patch, thereby exposing the fluid retaining layer. The protective layer 58 may be removed by peeling the protective layer from the adhesive layer. The sealing device, e.g., cap, is then removed from the sealed container 10, and the contents of the container are then be deposited onto the retaining portion of the adhesive layer 56 of the topical patch 50. As indicated above, a sufficient volume of the fluid composition is applied, where in many embodiments volume of fluid applied typically ranges from about 0.3 ml to about 2 ml, usually from about 0.5 ml to about 1.5 ml and more usually from about 0.6 ml to about 1 ml. If a second sealing device is disposed about the opening of the container, the user can utilize a suitable object to puncture or remove the second sealing device, wherein the fluid contents of the container may be deposited onto the retaining layer of the topical patch 50. Once the fluid composition has been deposited onto the retaining layer, the resultant prepared or loaded topical patch may then be administered to the subject in need thereof, e.g., by affixing the topical patch to the desired location of the subject or patient's body. The topical patch may be applied in an inconspicuous area, such as an arm or a leg.

As indicated above, the subject methods are methods of topically delivering DTH inducing agents, e.g., DNCB, to a host or subject in need thereof. By “topical delivery” it is meant delivery via absorption through the skin. In using the loaded topical patch to topically administer a DTH inducing agent to the host, the topical patch is applied to the skin after preparation of the topical patch in accordance with the present invention, as described above.

The patch may be administered to any convenient topical site. Topical sites of interest include, but are not limited to: arms, leg, torso, etc. The surface area that is covered by the topical patch preparation following application must be sufficient to provide for the desired amount of agent administration, and in many embodiments ranges from about 1 to 200 cm², and in many embodiments from about 10 to 100 cm², usually from about 20 to 50 cm², e.g., 25 cm². In practicing the subject methods, a topical patch may be applied a single time or a plurality of times over a given time period, e.g., the course of the disease condition being treated, where the dosing schedule when a plurality of patches are administered over a given time period may be daily, weekly, biweekly, monthly, etc.

The topical patch is maintained in contact with the skin for a period of time sufficient to deliver an effective or therapeutic amount of DTH to the patient. In many embodiments, the period of time required to deliver the desired amount of agent is short, generally not exceeding about 6 hours, e.g., not more than about 3 hours, including not more than about 1 hour, such as not more than about 60 minutes, not more than about 30 minutes and in some embodiments not exceeding about 15 minutes. The period of time during which the preparation is maintained at the application site depends on the nature of the composition and the subject, e.g., their sensitivity to the active agent, but is generally at least about 1 minute, and often at least about 5 minutes.

Utility

The above-described fluid compositions, patches prepared therefrom and methods for using the same, find use in any application in which the topical administration of an DTH inducing agent to a host is desired. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

In many embodiments, the subject methods find use in the treatment of a disease condition. By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as viral load or side effects associated therewith. As such, treatment also includes situations where the pathological condition, or at least symptoms associated therewith, are completely inhibited, e.g., prevented from happening, or stopped, e.g. terminated, such that the host no longer suffers from the pathological condition, or at least the symptoms that characterize the pathological condition. As such, treatment includes both curing and managing a disease condition.

In many embodiments, the disease condition that is treated according to the subject methods is one that is a chronic disease. Chronic diseases of interest include, but are not limited to: chronic fatigue syndrome, systemic lupus erythematosus, leprosy, leishmaniasis, diseases associated with the presence of intracellular pathogenic agents (e.g., viruses, bacteria), such as cytomegalovirus, Candida, Cryptococcus, Penumocystis carinii, and the like.

Of particular interest is the use of the subject methods in the treatment, e.g., management, of immunocompromising disease conditions, and particularly HIV associated disease conditions, e.g., AIDS. Treatment in the context of HIV associated diseases means improvement of quality of life, e.g., via reduction in one or more symptoms, the occurrence of opportunistic infections, etc. In terms of quantifiable parameters associated with HIV disease conditions, the subject invention finds use in reducing viral load(Surrogate maker) and/or increasing the population of natural killer cells, while varying the population of at least one of CD4(Surrogate maker) cells and CD8 cells. Such changes in quantifiable parameters are achievable with application times that do not exceed 15 minutes in length. The following practical and comparative examples are offered by way of illustration and not by way of limitation.

Experimental

Practical and comparative examples are given below, but the manufacturing method is not limited thereby.

### I. PRACTICAL EXAMPLES

A. Practical Example 1-10% DNCB/isopropyl myristate solution DNCB 20 g was uniformly dissolved in isopropyl myristate 180 g (w/w), after which the resulting DNCB fluid composition was packed in TPL-419 aluminum tubes (capacity: 2 g; inner layer: polyamide-imide, Takeuchi Press Industries Japan) in quantities of 2 g each; the tubes were then completely sealed, and stored in a thermostatic chamber at 40 degrees Celsius and at a humidity of 75%.

### B. Practical Example 2

0.02% DNCB/Isopropyl Myristate Solution

DNCB 0.04 g was uniformly dissolved in isopropyl myristate 199.96 g (w/w), after which the resulting DNCB fluid composition was packed in TPL-419 aluminum tubes (capacity: 2 g; inner layer: polyamide-imide, Takeuchi Press Industries Japan) in quantities of 2 g each; the tubes were then completely sealed, and stored in a thermostatic chamber at 40 degrees Celsius and at a humidity of 75%.

### C. Practical Example 3

10% DNCB/Crotamiton Solution

DNCB 20 g was uniformly dissolved in crotamiton 180 g (w/w), after which the resulting DNCB fluid composition was packed in TPL-419 aluminum tubes (capacity: 2 g; inner layer: polyamide-imide, Takeuchi Press Industries Japan) in quantities of 2 g each; the tubes were then completely sealed, and stored in a thermostatic chamber at 40 degrees Celsius and at a humidity of 75%.

### D. Practical Example 4

0.02% DNCB/Crotamiton

DNCB 0.04 g was uniformly dissolved in crotamiton 199.96 g (w/w), after which the resulting DNCB fluid composition was packed in TPL-419 aluminum tubes (capacity: 2 g; inner layer: polyamide-imide, Takeuchi Press Industries Japan) in quantities of 2 g each; the tubes were then completely sealed, and stored in a thermostatic chamber at 40 degrees Celsius and at a humidity of 75%.

II. Stability Data

Stability data experiments were performed with a sample size of three for each practical example. The experiments were performed in an environment of 40 degrees Celsius and 75% humidity. The results are shown as a percentage relative to the initial value in Table 1.

TABLE 1

| | | After 1 | After 2 | After 3 | After 4 | After 5 | After 6 |
|---|---|---|---|---|---|---|---|
| | Baseline | Month | Months | Months | Months | Months | Months |
| Practical | 100% | 99.8% | 99.2% | 100.02% | 99.5% | 99.0% | 98.7% |
| Example 1 | | | | | | | |
| Practical | 100% | 100.01% | 98.7% | 99.4% | 99.8% | 99.0% | 99.0% |
| Example 2 | | | | | | | |
| Practical | 100% | 99.9% | 99.6% | 100.03% | 100.01% | 98.7% | 99.2% |
| Example 3 | | | | | | | |
| Practical | 100% | 99.5% | 99.2% | 99.7% | 99.8% | 99.0% | 99.5% |
| Example 4 | | | | | | | |

III. Discussion

Based on Table 1, supra, it is demonstrated that it is possible to ensure adequate stability of a fluid composition including DNCB at 40 degrees Celsius in each practical example. Table 1 also demonstrates that good stability is obtained at both a concentration of 10% and at a concentration of 0.02%, so the stability is not dependent on the DNCB concentration. Thus, this makes it possible that DNCB patch preparations according to the present invention could be used in tropical regions and countries, such as Africa. Furthermore, applying a topical patch prepared in accordance with the present invention to a patient's skin for short period of time allows AIDS to be treated without a change in lifestyle of the patient, who is therefore able to lead exactly the same lifestyle as a healthy person. In addition, concomitant use with other HIV therapeutic medications is also quite possible. Research has been conducted for some time into the adverse events of DNCB, and there have not been any reports to date of cases of life-threatening adverse events, such as carcinogenicity. Moreover, the DNCB water-soluble topical patch of the present invention is only applied once a week, so treatment is possible at a cost of approximately $600.00 per person per year, making the patch of the present invention less expensive than other HIV therapeutic medications and making it possible to use it in developing countries as well.

It is evident from the above results and discussion that the subject invention provides for a number of advantages in the delivery of DTH inducing agents. The subject storage stable fluid compositions and kits that include the same provide an efficient and effective way to provide topical DTH preparations for use in a wide variety of different environments. The subject preparations represent a low cost way of treating many disease conditions, including AIDS. As such, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. An elevated temperature storage stable fluid composition of a delayed type hypersensitivity inducer agent, said composition comprising:
said delayed type hypersensitivity inducer agent present in a non-volatile organic solvent that is substantially inert to said delayed type hypersensitivity inducer agent at temperatures above about 40° C.

2. The storage stable fluid composition according to claim 1, wherein said delayed-type hypersensitivity inducer agent is 1-Chloro-2,4 Dinitrobenzene (DNCB).

3. The storage stable fluid composition according to claim 1, wherein said solvent does not contain hydroxyl groups.

4. The storage stable fluid composition according to claim 1, wherein said solvent does not contain metallic ions.

5. The storage stable fluid composition according to claim 1, wherein said solvent is an ester.

6. The storage stable fluid composition according to claim 1, wherein said solvent is chosen from: diisopropyl adipate, isopropyl myristate, diethyl sebacate, diethyltoulamide, crotamiton, and paraffin.

7. The storage stable fluid composition according to claim 1, wherein said composition is present in a sealed container.

8. The storage stable fluid composition according to claim 7, wherein said fluid composition is stable at temperatures above about 40° C. for at least about 100 days.

9. The storage stable fluid composition according to claim 8, wherein said fluid composition is stable at temperatures above about 40° C. for at least about 1 year.

10. A sealed container that contains an elevated temperature storage stable fluid composition of a delayed type hypersensitivity inducer agent present in a non-volatile organic solvent that is substantially inert to said delayed type hypersensitivity inducer agent at temperatures above about 40° C.

11. The sealed container according to claim 10, wherein said container is a single dosage container.

12. The sealed container according to claim 10, wherein said container comprises a material that is substantially inert to said fluid composition.

13. The sealed container according to claim 12, wherein said substantially inert material is a metallic material.

14. The sealed container according to claim 12, wherein said substantially inert material is a polymeric material.

15. The sealed container according to claim 10, wherein said container is generally tubular in shape and includes a removable sealing portion.

16. A kit for making a topical preparation of a delayed type hypersensitivity inducer agent, said kit comprising:
(a) an elevated temperature storage stable fluid composition of said delayed type hypersensitivity inducer agent present in a non-volatile organic solvent that is substantially inert to said delayed type hypersensitivity inducer agent at temperatures above about 40° C.; and
(b) an unloaded topical patch preparation having a fluid composition retaining region.

17. The kit according to claim 16, wherein said fluid composition is present in a sealed container.

18. The kit according to claim 16, wherein said sealed container is a single dosage container.

19. The kit according to claim 16, wherein said delayed-type hypersensitivity inducer is 1-Chloro-2,4 Dinitrobenzene (DNCB).

20. The kit according to claim 16, wherein said topical patch preparation includes:
(a) a backing;
(b) an adhesive layer; and
(c) said fluid composition retaining region.

21. The kit according to claim 16, wherein said kit further includes instructions, or means for obtaining the same, recorded onto a substrate, wherein said instructions are for preparing a topical patch preparation by applying said fluid composition to said fluid retaining region of said unloaded topical patch.

22. A method of preparing a topical patch for use, said method comprising:
(a) providing an elevated temperature storage stable fluid composition of said delayed type hypersensitivity inducer agent present in a non-volatile organic solvent that is substantially inert to said delayed type hypersensitivity inducer agent at temperatures above about 40° C.; and
(b) applying said fluid composition onto a fluid composition retaining region of an unloaded topical patch preparation to prepare said topical patch for use.

23. The method according to claim 22, wherein said providing step (a) comprises providing a sealed container of said fluid composition and opening said container.

24. The method according to claim 22, wherein said method further comprises removing said unloaded topical patch preparation from a protective container.

25. The method according to claim 23, wherein said container and said unloaded topical patch are present in a kit containment element.

26. A method of administering a topical preparation of a delayed type hypersensitivity inducer agent to a subject, said method comprising:
(a) preparing a topical patch according to the method of claim 22; and
(b) topically applying said prepared topical patch to said subject.

27. A method of making the kit according to claim 16, said method comprising:
(a) producing an elevated temperature storage stable fluid composition of said delayed type hypersensitivity inducer agent present in a non-volatile organic solvent that is substantially inert to said delayed type hypersensitivity inducer agent at temperatures above about 40° C.;
(b) sealing said produced fluid composition into a container to produce a sealed container containing said fluid composition; and
(c) placing said sealed container and an unloaded topical patch preparation into a kit containment element to produce said kit.
